(19) United States
(12) Patent Application Publication
(10) Pub. No.: US 2024/0307643 A1
(43) Pub. Date: Sep. 19, 2024

(54) GAUZE PLACEMENT APPARATUSES AND RELATED METHODS

(71) Applicant: CarJess Medical Supply, Inc., Raeford, NC (US)

(72) Inventor: Juan Carlos Lopez Jimenez, III, Raeford, NC (US)

(21) Appl. No.: 18/274,842

(22) PCT Filed: Aug. 30, 2022
(86) PCT No.: PCT/US2022/042062

§ 371 Date: Jul. 28, 2023

Related U.S. Application Data
(60) Provisional application No. 63/238,480, filed on Aug. 30, 2021.

(51) Int. Cl. A61M 16/04; A61F 15/00

(52) U.S. Cl. CPC A61M 16/047; A61F 15/005

(57) ABSTRACT

Gauze placement apparatuses and related methods are disclosed herein. The gauze placement apparatus can include a lower fork extension that can have a fork portion and first and second lower prongs aligned with each other in a second direction that is transverse to a first direction in which an upper handle is oriented and an upper fork extension that can have a fork portion and first and second upper prongs aligned with each other in the second direction that is transverse to the first direction in which a lower handle is oriented. The upper handle and the lower fork extension being pivotally connected to the lower handle and the upper fork extension such that the upper handle resides above the lower handle and the upper fork extension resides above the lower fork extension. When the upper handle and lower handle are moved in a diverging direction, the upper and lower fork extensions move apart from each other to an open position. When the upper handle and lower handle are moved in a converging direction, the upper and lower fork extensions move toward each other to a closed position. The first and second lower prongs of the lower fork extension can be configured to receive a first folded gauze such that a slit within the gauze resides between the first and second lower prongs. The upper and lower fork extensions can be configured to hold the first folded gauze when in the closed position.

### RELATED APPLICATIONS

The present patent application claims the benefit of PCT International Patent Application Serial. No. PCT/US2022/042062, filed Aug. 30, 2022, and U.S. Provisional Patent Application Ser. No. 63/238,480, filed Aug. 30, 2021, to which PCT International Patent Application Serial. No. PCT/US2022/042062 claims priority and benefit. The disclosures of both patent applications referenced above are incorporated herein by reference in their entireties.

### TECHNICAL FIELD

The present subject matter relates to gauze placement apparatuses and related methods. In particular, the present subject matter relates to gauze placement apparatuses capable of gripping a piece of gauze and placing the piece of gauze on a patient around an opening on the patient in which medical equipment resides. For an example, the present subject matter relates to gauze placement apparatuses capable of gripping a piece of gauze and placing the piece of gauze on a patient around an opening of a tracheostomy on the patient and the tracheostomy equipment.

### BACKGROUND

Tracheostomies are openings surgically created at the front of the neck in order for a tube to be inserted into the windpipe to help one breathe. A tracheostomy may be necessary when built up fluid cannot be cleared from one's airways because of preexisting symptoms like long-term pain, muscle weakness, or paralysis. If necessary, the tube can be connected to an oxygen supply and a breathing machine called a ventilator. When this is the case, the opening is covered by equipment and medical supplies such as gauze. It is imperative that the gauze is regularly swapped out with fresh gauze to keep the opening clean and sterile. However, placing gauze with the equipment in place can be difficult, especially considering the sensitive area of the patient's neck. One might also want to avoid having to touch the patient's skin completely for sanitary reasons.

As such, a need exists for a gentle and efficient way of placing gauze on a patient around tracheostomy equipment in a manner that is both clean and sterile and to provide apparatuses that can aid in accomplishing this task.

### SUMMARY

The present subject matter relates to gauze placement apparatuses for placing gauzes on a patient. In particular, the present subject matter relates to gauze placement apparatuses capable of gripping a piece of gauze and placing the piece of gauze on a patient around an opening on the patient in which medical equipment resides. For an example, the present subject matter relates to gauze placement apparatuses capable of gripping a piece of gauze and placing the piece of gauze on a patient around an opening of a tracheostomy on the patient and the tracheostomy equipment. Methods related to the use of the gauze placement apparatuses disclosed herein are also provided.

Thus, it is an object of the presently disclosed subject matter to provide gauze placement apparatuses for placing gauzes on a patient as well as methods related thereto. While one or more objects of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

A full and enabling disclosure of the present subject matter including the best mode thereof to one of ordinary skill in the art is set forth more particularly in the remainder of the specification, including reference to the accompanying figures, in which:

FIG. 1 illustrates a bottom perspective view of an embodiment of a gauze placement apparatus according to the present subject matter in an open position;

FIG. 2 illustrates a side perspective view of the embodiment of the gauze placement apparatus according to FIG. 1 in a closed position;

FIG. 3 illustrates an exploded view of the embodiment of the gauze placement apparatus according to FIG. 1;

FIG. 4 illustrates side plan view of the embodiment of the gauze placement apparatus according to FIG. 1 with the gauze placement apparatus in an open position;

FIG. 5 illustrates side plan view of the embodiment of the gauze placement apparatus according to FIG. 1 with the gauze placement apparatus in a closed position;

FIG. 6 illustrates a top perspective view of an embodiment of a gauze placement apparatus with a gauze being placed on a lower set of prongs according to the present subject matter;

FIG. 7 illustrates a top perspective view of the embodiment of the gauze placement apparatus according to FIG. 6 with the top set of prongs being clamped down on the gauze being placed on the lower set of prongs according to the present subject matter;

FIGS. 8 and 9 illustrate a top perspective view of the embodiment of a gauze placement apparatus according to FIG. 6 placing the gauze on a patient around tracheostomy equipment inserted into and resides in the patient according to the present subject matter;

FIG. 10 illustrates a top perspective view of an embodiment of a gauze placement apparatus with a gauze being placed on a lower set of prongs and a gauze being placed on an upper set of prongs according to the present subject matter; and

FIG. 11 illustrates a top perspective view of another embodiment of a gauze placement apparatus according to the present subject matter.

Repeat use of reference characters in the present specification and drawings is intended to represent the seam or analogous features or elements of the present subject matter.

### DETAILED DESCRIPTION

Reference now will be made to the embodiments of the present subject matter, one or more examples of which are set forth below. Each example is provided by way of an explanation of the present subject matter, not as a limitation. In fact, it will be apparent to those skilled in the art that various modifications and variations can be made in the present subject matter without departing from the scope or spirit of the present subject matter. For instance, features illustrated or described as one embodiment can be used on another embodiment to yield still a further embodiment. It is to be understood by one of ordinary skill in the art that the present discussion is a description of exemplary embodiments only, and is not intended as limiting the broader aspects of the present subject matter, which broader aspects are embodied in exemplary constructions.

Although the terms first, second, right, left, front, back, top, bottom, upper lower, vertical, horizontal, etc. may be used herein to describe various features, elements, components, regions, layers and/or sections, these features, elements, components, regions, layers and/or sections should not be limited by these terms. These terms are only used to distinguish one feature, element, component, region, layer or section from another feature, element, component, region, layer, or section. Thus, a first feature, element, component, region, layer, or section discussed below could be termed a second feature, element, component, region, layer, or section without departing from the teachings of the disclosure herein. Additionally, such terms as right, left, front, back, top, bottom, upper lower, vertical, horizontal, etc. are simply describing the relative position or movement of the features or elements to each other and do not necessarily mean an absolute position or movement since the relative position or movement depends upon the orientation of the device to the viewer and/or user.

Similarly, when a feature or element is being described in the present disclosure as “on” or “over” another feature or element, it is to be understood that the features or elements can either be directly contacting each other or have another feature or element between them, unless expressly stated to the contrary. As above, these terms are simply describing the relative position of the features or elements to each other and do not necessarily mean “on top of” since the relative position above or below depends upon the orientation of the device to the viewer.

Embodiments of the subject matter of the disclosure are described herein with reference to schematic illustrations of embodiments that may be idealized. As such, variations from the shapes and/or positions of features, elements, or components within the illustrations as a result of, for example but not limited to, user preferences, manufacturing techniques and/or tolerances are expected. Shapes, sizes and/or positions of features, elements or components illustrated in the figures may also be magnified, minimized, exaggerated, shifted, or simplified to facilitate explanation of the subject matter disclosed herein. Thus, the features, elements or components illustrated in the figures are schematic in nature and their shapes and/or positions are not intended to illustrate the precise configuration of the subject matter and are not necessarily intended to limit the scope of the subject matter disclosed herein unless it specifically stated otherwise herein.

It is to be understood that the ranges and limits mentioned herein include all ranges located within the prescribed limits (i.e., subranges). For instance, a range from about 100 to about 200 also includes ranges from 110 to 150, 170 to 190, 153 to 162, and 145.3 to 149.6. Further, a limit of up to about 7 also includes a limit of up to about 5, up to 3, and up to about 4.5, as well as ranges within the limit, such as from about 1 to about 5, and from about 3.2 to about 6.5.

The present subject matter relates to is a gauze placement apparatus for medical treatment of tubular or other inserts that extend outward from a body of a patient. The present invention attempts to provide medical personnel with a more effective way to place gauze around the opening for an insert, for example, an opening of a tracheostomy, on a patient. The present invention attempts this with the various components involved.

Thus, the present disclosure relates to gauze placement apparatuses for placement of gauze on a patient around a portion of medical equipment that has been inserted into the patient. For example, the same or different embodiments of the gauze placement apparatuses can be used for placing gauze on a patient around equipment inserted into a body of patient, such as stoma equipment, tracheostomy equipment, colostomy bag equipment, I.V. equipment, or the like. For example, the gauze placement apparatus comprises a pair of handles and a pair of fork extensions connected to the respective handles. One handle is positioned above the other handle. The pair of fork extensions can be positioned so each forked extension is vertically in line with the other. Each forked extension can also include a fork portion and a pair of prongs extending from the fork portion. The pair of prongs on each forked extension can be horizontally in line with the other. The pair of handles can be described as a first handle and a second handle. Similarly, the pair of forked extensions can be considered a first forked extension and a second forked extension. The pair of handles and the pair of forked extensions can be pivotally connected such that the first handle can reside above the second handle and the first forked extension can reside above the second forked extension. As the pair of handles is squeezed together, the pivotal connection allows the pair of forked extensions to be squeezed together as well. As the pair of handles is separated, the pair of forked handles is separated as a result.

Such a gauze placement apparatus can make it easy to place a gauze such as a slit gauze on a patient around equipment that extends out of the body of the patient. The gauze placement apparatus can also increase the speed at which gauze placement and/or replacement on a patient around medical equipment on or partially inserted into the patient can occur. As used herein, the term “around” means being on at least a side of the medical equipment, but does not mean that the gauze needs to completely encircle the medical equipment. Using the gauze placement apparatus to place a gauze in such areas on a patient is much faster and easier that a medical professional doing it by hand. An example of the use of the gauze placement apparatus can be described with reference to the placement of a slit gauze on the neck of a patient with a tracheostomy. The user of the device can separate the pair of handles and thus separate the pair of forked extensions. A piece of slit gauze, which can be a folded gauze with a slit on a folded side of the folded gauze, can then be placed between the first forked extension and the second forked extension. Once in place with the slit of the slit gauze between the prongs of the forked extensions, the pair of handles can be squeezed in order to secure the gauze between the pair of forked extensions.

With the patient lying on the patient's back, the user of the device can move the device up past the collarbones of the patient to the location of the hole created for the tracheostomy. The prongs of the pair of forked extensions are configured to be wide enough apart for a slit gauze to be grasped and to permit insertion of the prongs and the slit gauze beneath any tracheostomy equipment, and around the any equipment insertion extending from the neck of the patient. Once the gauze is in the most effective spot, the user can release tension on the pair of handles, effectively releasing the grip of the pair of forked extensions on the gauze. The gauze can be secured in the necessary spot while the user retracts the gauze placement apparatus from any equipment it may have been beneath. The gauze placement apparatus can also be used to remove gauze in a similar fashion, just using steps opposite to the steps described above. In some embodiments, the gauze placement apparatus can comprise a metal. In some embodiments, the gauze placement apparatus can comprise a polymer, such as a plastic material, for instance, a thermoplastic material. In some embodiments, the gauze placement apparatus can comprise combination of one or more metals and/or one or more plastic materials. In some embodiments, the gauze placement apparatus can be manufactured using a polymer, such as a flexible plastic material, so that if the user accidentally pushes the device to hard against the patient, the device will bend rather than potentially harm the patient.

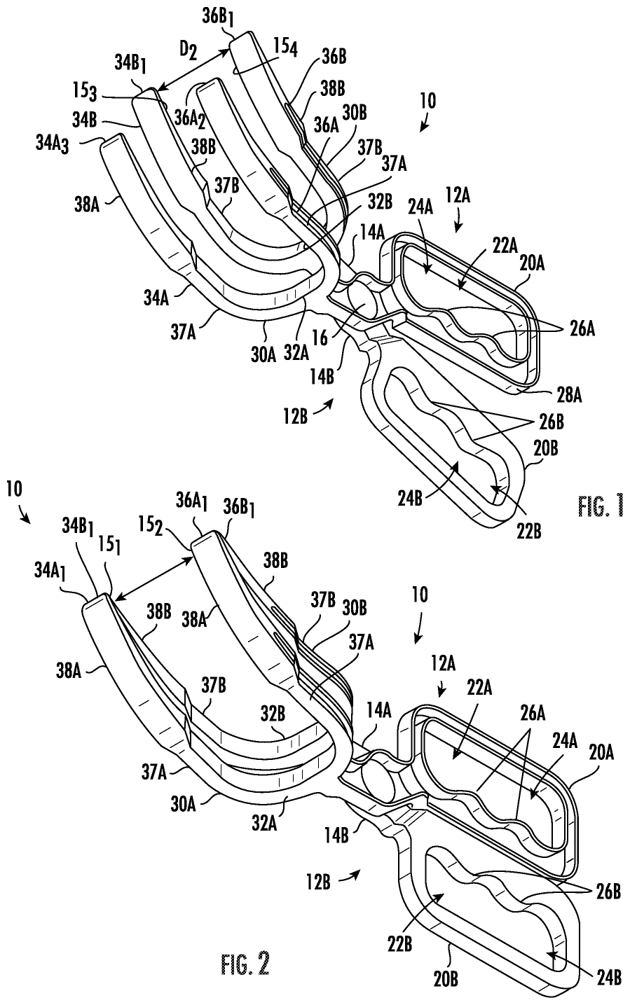

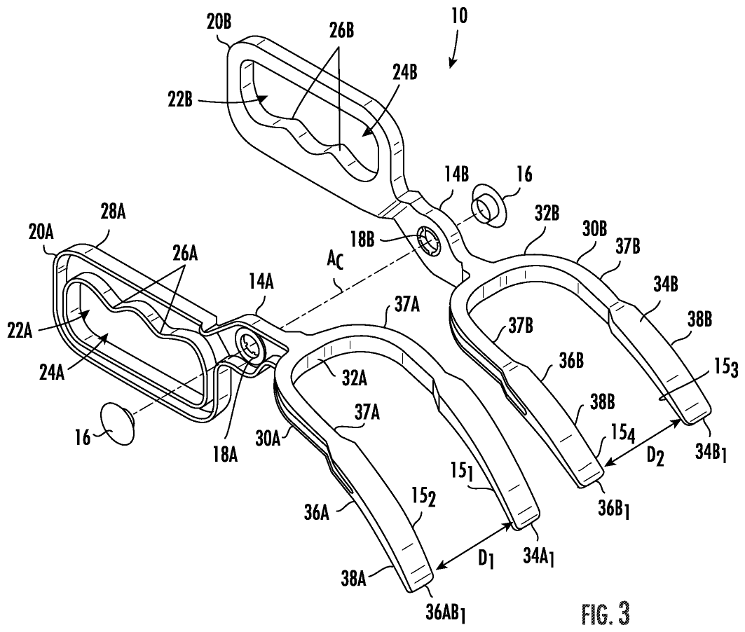

Referring to FIGS. 1-10, a gauze placement apparatus, generally designated 10, can be provided. The gauze placement apparatus 10 can comprise an upper handle 20A connected to a lower fork extension 30A and a lower handle 20B connected to an upper fork extension 30B. The upper handle 20A and the lower fork extension 30A can be pivotally connected to the lower handle 20B and the upper fork extension 30B such that the upper handle 20A resides above the lower handle 20B and the upper fork extension 30B resides above the lower fork extension 30A. In this manner, when the upper handle 20A and lower handle 20B are moved in a diverging direction, the upper and lower fork extensions 30B, 30A move apart from each other to an open position as shown in FIG. 1. When the upper handle 20A and lower handle 20B are moved in a converging direction, the upper and lower fork extensions 30B, 30A move toward each other to a closed position as shown in FIG. 2.

For example, in some embodiments as shown in FIG. 1-3, the gauze placement apparatus 10 can comprise a first blade member 12A that can comprise the upper handle 20A connected by a linking shaft 14A to the lower fork extension 30A and a second blade member 12B that can comprise the lower handle 20B connected a first blade member 14B that can comprise the upper fork extension 30B. The first blade member 12A and second blade member 12B can be pivotally jointed together by a connector 16 that acts as a fulcrum to permit the first blade member 12A and second blade member 12B to partially rotate, or pivot about an axis A_{C} of the connector 16. For example, the first blade member 12A and second blade member 12B can be connected to each other along the linking shafts 14A, 14B. The connector 16 can be a variety of fasteners that permit such a pivoting action of the first blade member 12A and second blade member 12B relative to each other. For example, the connector 16 can comprise, rivets, snap rivets, pivot screws and nuts, or the like. In such embodiments, the first blade member 12A and second blade member 12B can each have a fastening aperture 18A, 18B, which can be in the linking shafts 14A, 14B for receiving the connector 16. Additionally, in some embodiments, not shown, the connector 16 can comprise snap fasteners formed on the sides of the linking shafts 14A, 14B that are configured to engage each other in a manner that permits the pivoting movement of the first blade member 12A and second blade member 12B relative to each other.

The linking shafts 14A, 14B can be angled or curved relative to the handles 20A, 20B and the fork extensions 30A, 30B such the upper handle 20A resides above lower handle 20B and the upper fork extension 30B is above the lower fork extension 30A. This configuration of the linking shafts 14A, 14B and first blade member 12A and second blade member 12B can facilitate the opening and closing movement of the gauze placement apparatus 10. In particular, the angled or curved linking shafts 14A, 14B can ensure proper placement of the handles 20A, 20B and the fork extensions 30A, 30B relative to one another.

The handles 20A, 20B can each comprise an insert aperture 22A, 22B to permit the insertion of at least a portion of a hand such as one or more fingers or a thumb into the respective insertion aperture 22A, 22B to move the upper and lower fork extensions between the open position to the closed position. For example, the handles 20A, 30B can each comprise a hand insert 24A, 24B that permits insertion of multiple fingers in the hand insert 24A, 24B to increase versatility of the gauze placement apparatus 10. Each hand insert 24A, 24B can have finger placement ridges 26A, 26B to facilitate the grip of the user on the handles 20A, 20B. Further, in some embodiments, the handles 20A, 20B can be aligned in a first direction A₁, herein referenced as a vertical direction, so that the handles 20A, 20B can be easily and ergonomically moved by either a right hand or a left hand of the user. For example, in some embodiments, the handles 20A, 20B and/or the linking shafts 14A, 14B can be configured so that a side perimeter wall 28A of handle 20A can abut a side perimeter wall 28B of handle 20B when the handles 20A, 20B and fork extensions 30A, 30B are in a closed position.

The upper and lower fork extensions 30B, 30A can each have similar features that can be aligned with each other as well. The lower fork extension 30A can comprise a lower fork portion 32A. The lower fork extension 30A can also comprise a first lower prong 34A and a second lower prong 36A that extend outward from the fork portion 32A away from the handles 20A, 20B. As shown in FIGS. 1-3, the first lower prong 34A and a second lower prong 36A aligned with each other in a second direction A₂, herein referenced as a horizontal direction, that is transverse to the first direction A₁ in which the upper handle is oriented. Similarly, the upper fork extension 30B can comprise an upper fork portion 32B. The upper fork extension 30B can also comprise a first upper prong 34B and a second upper prong 36B that extend outward from the upper fork portion 32B away from the handles 20A, 20B. As shown in FIGS. 1-3, the first upper prong 34A and a second upper prong 36A aligned with each other in the same second direction A₂, or horizontal direction, that is transverse to the first direction A₁ in which the lower handle is oriented.

Both the first and second lower prongs 34A, 36A of the lower fork extension 30A and the first and second upper prongs 34B, 36B of the upper fork extension 30B can be configured to hold gauzes and place the gauzes in a desired location on the patient. In particular, the first and second lower prongs 34A, 36A and the first and second upper prongs 34B, 36B can be configured to facilitate the placing split gauzes on a patient around medical equipment inserted in and extending from the body of the patient. To facilitate the placement of such split gauzes, the first and second lower prongs 34A, 36A can be separated from each other at a distance D₁ in the second direction A₂ that permits the first and second lower prongs 34A, 36A to be placed or inserted around a tube or other insert from medical equipment. Similarly, the first and second upper prongs 34B, 36B can be separated from each other at a distance D₂ in the second direction A₂ that permits the first and second upper prongs 34B, 36B to be placed or inserted around the tube or other insert from medical equipment. In some embodiments, the distance D₁ between the first and second lower prongs 34A, 36A can be similar to or the same as distance D₂ between the first and second upper prongs 34B, 36B. In some embodiments, the distance D₁ can be measured between an inner surface IS₁ of the first lower prong 34A and an inner surface IS₂ of the second lower prong 36A and the distance D₂ can be measured between an inner surface IS₃ of the first lower prong 34B and an inner surface IS₄ of the second lower prong 36B. The distances D₁ and D₂ can be dependent on the medical equipment with which the gauze placement device is intended to be used. For some intended uses, the embodiment of the gauze placement apparatus 10 can have smaller distances D₁ and D₂. For some intended uses, the embodiment of the gauze placement apparatus 10 can have larger distances D₁ and D₂. For example, an embodiment of the used for pediatric tracheostomies can have smaller dimensions including smaller distances D₁ and D₂ than an embodiment of gauze placement apparatus 10 used in conjunction with adult tracheostomies.

In some embodiments, the distance D₁ and the distance D₂ are equal. For example, in some embodiments, the distances D₁ and D₂ can be between about 1.25 inches and about 4 inches. In some embodiments, the distances D₁ and D₂ can be between about 1.50 inches and about 2.5 inches. In some embodiments, the distances D₁ and D₂ can be between about 1.50 inches and about 2 inches.

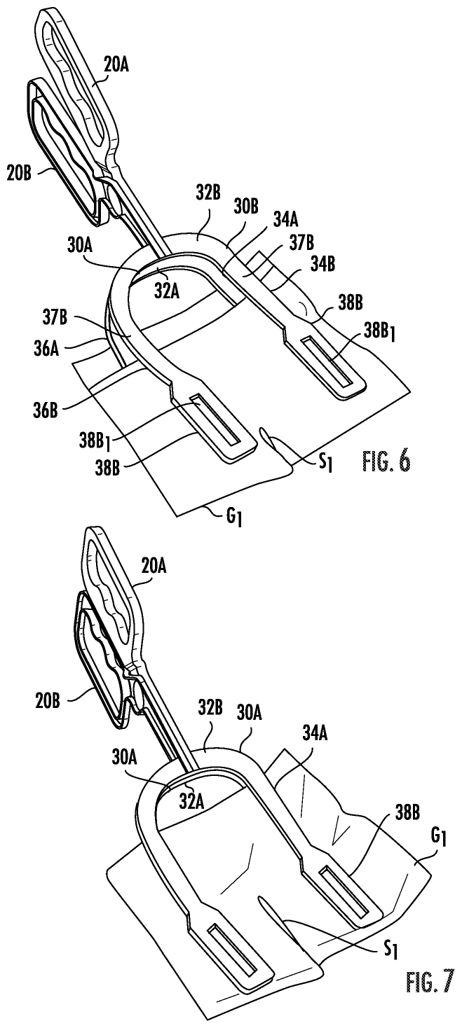

The manner in which the first and second lower prongs 34A, 36A and the first and second upper prongs 34B, 36B are configured, including the distances D₁ and D₂, can facilitate the holding and placement of a gauze. For example, when applying a gauze on a patient around a tube or an insert of medical equipment that is inserted into the body of a patient, a split gauze can be placed on either the first and second lower prongs 34A, 36A of the lower fork extension 30A or the first and second upper prongs 34B, 36B of the upper fork extension 30B or on both sets of prongs 34A, 36A and 34B, 36B. For example, the first and second lower prongs 34A, 36A of the lower fork extension 30A The first and second lower prongs 34A, 36A of the lower fork extension 30A can thus be configured to receive a first slit gauze G₁, i.e., a folded gauze with a slit S on a folded side of the folded gauze as shown in FIGS. 6 and 7. When the slit gauze G₁ is properly placed on the first and second lower prongs 34A, 36A of the lower fork extension 30A, the slit S in the split gauze G₁ will reside between the first and second lower prongs 34A, 36A and the upper and lower fork extensions 30B, 30A are configured to hold at least a portion of the first slit gauze G₁ when in the closed position. In particular, the first and second lower prongs 34A, 36A can each comprise ends 34A₁, 36A₁ against which the fold of the gauze G₁ can reside when the gauze G₁ is placed on the first and second lower prongs 34A, 36A to help hold the gauze G₁ taut and in proper position. The ends 34A₁, 36A₁ can be blunt ends that can protect both the gauze G₁ and the patient.

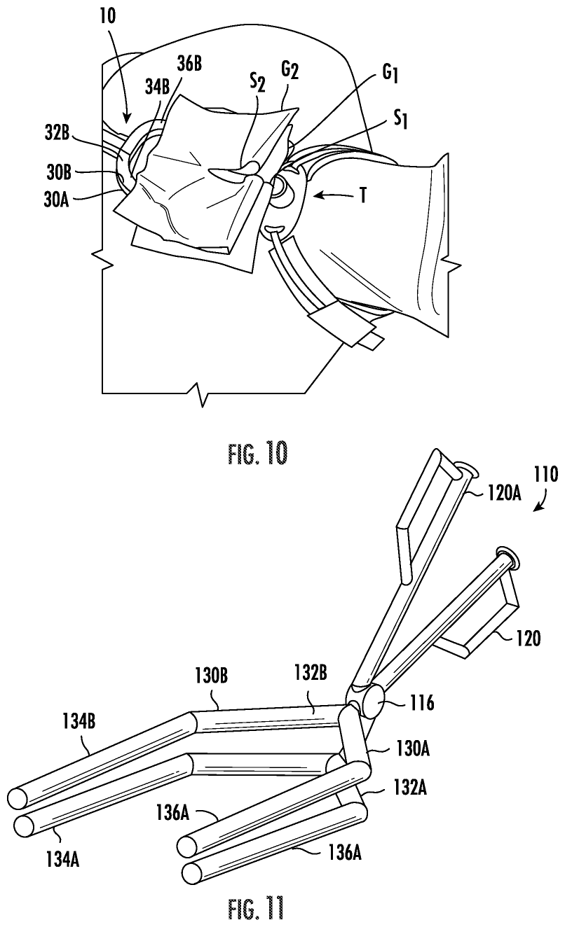

Similarly, the first and second upper prongs 34B, 36B of the upper fork extension 30B can be configured to receive a second slit gauze G₂, i.e., a folded gauze with a slit S on a folded side of the folded gauze as shown in FIG. 10. When the slit gauze G₂ is properly placed on first and second upper prongs 34B, 36B of the upper fork extension 30B, the slit S in the split gauze G₁ will reside between the first and second upper prongs 34B, 36B and the upper and lower fork extensions 30B, 30A are configured to hold the second slit gauze G₁ when in the closed position. As with the lower prongs 34A, 36A, the first and second upper prongs 34B, 36B can each comprise ends 34B₁, 36B₁ against which the fold of the gauze G₁ can reside when the gauze G₁ is placed on the prongs 34B₁, 36B₁. The ends 34B₁, 36B₁ can be blunt ends that can protect both the gauze G₁ and the patient.

The first and second lower prongs 34A, 36A and the first and second upper prongs 34B, 36B can be configured in different ways. In some embodiments, the first and second lower prongs 34A, 36A and the first and second upper prongs 34B, 36B can be the same width from the fork portions 32A, 32B to the respective blunt ends 34A₁, 34B₁, 36A₁, 36B₁. In some embodiments as shown, the first and second upper prongs 34B, 36B of the upper fork extension 30B each can comprise a shank 37B and an insertion tip 38B. Similarly, the first and second lower prongs 34A, 36A of the lower fork extension 30A each can comprise shank 37A and an insertion tip 38A. For embodiments that include shanks 37A, 37B and insertion tips 38A, 38B, the shanks 37A, 37B and the insertion tips 38A, 38B can comprise different shapes, sizes, and lengths. In some embodiments, the insertion tips 38A, 38B can be longer than the shanks 37A, 37B. In some embodiments, the shanks 37A, 37B can be longer than the insertion tips 38A, 38B. In some embodiments, the insertion tips 38A, 38B can be 1.7 inches or more in length. In some embodiments, the insertion tips 38A, 38B can be between about 2 inches and about 4 inches in length. In some embodiments, the insertion tips 38A, 38B can be about the same length as a folded gauze as measured from the fold in the gauze to the ends of the gauze. In some embodiments, the insertion tips 38A, 38B can be about 4 inches in length. In some embodiments, the insertion tips 38A, 38B can have apertures 38A₁, 38B₁ therein as shown in FIGS. 6 and 7.

As shown in FIGS. 1, 2 and 4-5, in some embodiments, the first upper prong 34B aligns with the first lower prong 34A in the first direction A₁ that is traverse to the second direction A₂ in which the first and second upper prongs 34B, 36B are aligned. Similarly, the second upper prong 36B also aligns with the second lower prong 36A in the first direction A₁ that is traverse to the second direction A₂ in which the first and second upper prongs 34B, 36B are aligned. As shown in FIG. 5, the insertion tip 38B of the first upper prong 34B and the insertion tip 38A of the first lower prong 34A can converge in the closed position and the insertion tip 38B of the second upper prong 36B and the insertion tip 38A of the second lower prong 36A converge in the closed position to facilitate insertion of one or more gauzes G₁, G₂ between a patient and a portion of tracheostomy equipment. In some such embodiments, the shank 37B of the first upper prong 34B and the shank 37A of the first lower prong 34A can be spaced apart in the closed position and the shank 37B of the second upper prong 36B and the shank 37A of the second lower prong 36A are spaced apart in the closed position creating a separation space SS between portions of the upper fork extension 30B and the lower fork extension 30A.

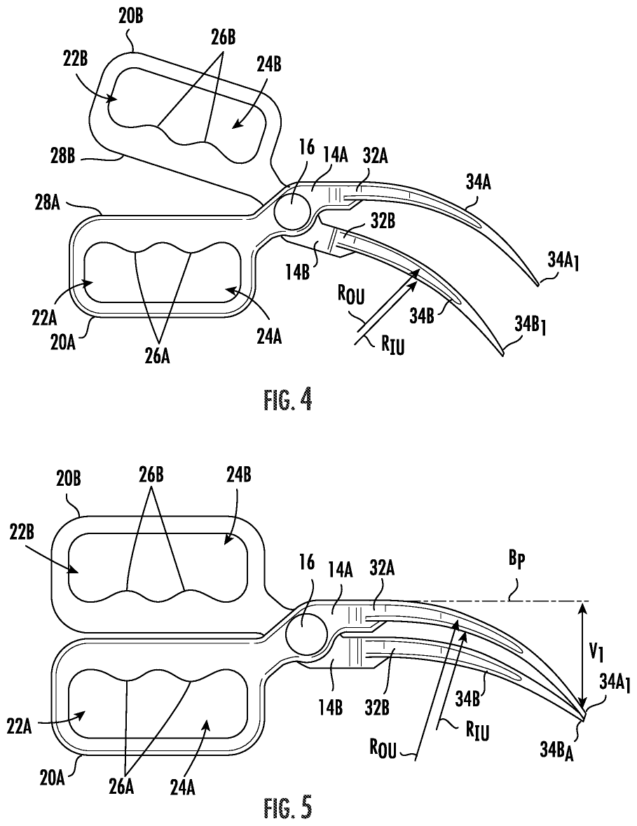

As shown in FIGS. 1-10, the first and second lower prongs 34A, 36A and the first and second upper prongs 34B, 36B of the gauze placement apparatus 10 can be curved to facilitate placement of gauzes on a patient around medical equipment, such a s tracheostomy equipment, that has been inserted into an incision or opening within the patient without disturbing the medical equipment or the patient. In particular, the first and second lower prongs 34A, 36A of the lower fork extension 30A and the first and second lower prongs 34B, 36B of the upper fork extension 30B can curve upward to facilitate insertion of one or more gauzes around a tube inserted into a body B of a patient P. As shown in FIGS. 4 and 5, to form the curvature of the prongs 34A, 36A, 34B, 36B, the first and second lower prongs 34A, 36A of the lower fork extension 30A each can comprise an inner radius of curvature R_{IL} and an outer radius of curvature R_{OL} and the first and second upper prongs 34B, 36B of the upper fork extension 30B each can comprise an inner radius of curvature R_{IU} and an outer radius of curvature R_{OU}. The inner radii of curvature R_{IL} of the first and second lower prongs 34A, 36A are greater than the outer radii of curvature R_{IL} of first and second lower prongs 34A, 36A such that upper and lower outer surfaces of first and second lower prongs 34A, 36A converge toward blunt ends 34A₁, 36A₁ of the respective first and second lower prongs 34A, 36A to form the shape of the first and second lower prongs 34A, 36A. Similar, the inner radii of curvature R_{IU} of the first and second upper prongs 34B, 36B are greater than the outer radii of curvature R_{OU} of first and second upper prongs 34B, 36B such that upper and lower outer surfaces of first and second upper prongs 34B, 36B converge toward blunt ends 34B₁, 36B₁ of the respective first and second upper prongs 34B, 36B to form the shape of the first and second upper prongs 34B, 36B. Further, to form the pronged section of the gauze placement apparatus 10, the outer radii of curvature R_{OU} of first and second upper prongs 34B, 36B are greater than the outer radii of curvature R_{OL} of first and second lower prongs 34A, 36A such that the first and second lower prongs 34A, 36A converge toward the respective first and second upper prongs 34B, 36B when the upper and lower fork extensions 30B, 30A are in the closed position.

In addition to the curvature of the prongs 34A, 36A, 34B, 36B of the upper and lower fork extensions 30B, 30A of the gauze placement apparatus 10, the prongs 34A, 36A, 34B, 36B can each have a curved length that also helps to facilitate placement of gauzes between medical equipment and the patient P and around a portion of the medical equipment that is inserted into the patient P. For example, in some embodiments, the outer radii of curvature R_{OL} of first and second lower prongs 34A, 36A and lengths of the first and second upper prongs 34B, 36B of the upper fork extensions 30B and the first and second lower prongs 34A, 36A of the lower fork extensions 30A are such that the blunt ends 34A₁, 36A₁ of the first and second lower prongs 34A, 36A and the blunt ends 34B₁, 36B₁ of the first and second upper prongs 34B, 36B can be inserted under portions of a tracheostomy equipment T and a slit gauze G₁, G₂ on the upper prongs placed around a tube T₁ of the tracheostomy equipment T with the base SB of a slit S of the slit gauze G₁, G₂ against the tube T₁ without the fork portion 32A of the lower fork extension or the lower handle 20B abutting the body B, i.e., the chest, of the patient P. In particular, the outer radii of curvature R_{OL} of first and second lower prongs 34A, 36A and lengths of the first and second upper prongs 34B, 36B and the first and second lower prongs 34A, 36A create a vertical displacement in the first direction A₁ of the blunt ends 34A₁, 36A₁ of the first and second lower prongs 34A, 36A and the blunt ends 34B₁, 36B₁ of the first and second upper prongs 34B, 36B from a base 32A_{B} of the lower fork portion 32A of the lower fork extensions 30A that can facilitate insertion of gauze between medical equipment and the patient P and around a portion of the medical equipment that is inserted into the patient P. For example, the first and second lower prongs 34A, 36A can curve upward from the fork portion 32A of the lower fork extension 30A such that a vertical distance V₁ as measured from a plane BP that extends from an outer surface of a base 32A_{B} of the fork portion 32A of the lower fork extension 30A to the blunt ends 34A₁, 36A₁ of first and second lower prongs 34A, 36A that can facilitate insertion of one or more gauzes by the gauze placement apparatus 10 between medical equipment and the patient P and around a portion of the medical equipment that is inserted into the patient P. In some embodiments, the vertical distance V₁ can be 1.5 inches or more. In some embodiments, the vertical distance V₁ can be 1.7 inches or more. In some embodiments, the vertical distance V₁ can be 1.9 inches or more. In some embodiments, the vertical distance V₁ can be 1.5 inches or more can be about 2 inches or more. In some embodiments, the vertical distance V₁ can be between about 1.5 inches and about 4 inches. In some embodiments, the vertical distance V₁ can be between about 2 inches and about 4 inches.

As stated above, different embodiments of the gauze placement apparatus 10 having assorted sizes and configurations of the prongs 34A, 36A, 34B, 36B can be used with different medical equipment, such as medical equipment used for stomas, colostomy bags, tracheostomy, or the like. Additionally, the gauze placement apparatus 10 can have different sizes depending on the size and type of patient. For example, an embodiment of the gauze placement apparatus 10 for a pediatric tracheostomy can be a smaller size from an embodiment of the gauze placement apparatus 10 for an adult tracheostomy. Referring to FIGS. 6-10, as an example use, an embodiment of the gauze placement apparatus 10 that can be used to place gauze between a patient P and tracheostomy equipment T is described. As shown in FIGS. 6-10, a gauze placement apparatus 10 can be provided that can comprise upper handle 20A connected to a lower fork extension 30A. The lower fork extension 30A can comprises a fork portion 32A and first and second lower prongs 34A, 36A aligned with each other in the second direction A₂ transverse to the first direction A₁ in which the upper handle 20A is oriented. Similarly, the gauze placement apparatus 10 can comprise a lower handle 20B connected to an upper fork extension 30B. The upper fork extension 30B can comprise a fork portion 32B and first and second upper prongs 34B, 36B aligned with each other in the second direction A₂ that is transverse to the first direction A₁ in which the lower handle 20B is oriented. The upper handle 20A and the lower fork extension 30A can be pivotally connected to the lower handle 20B and the upper fork extension 30B such that the upper handle 20A resides above the lower handle 20B and the upper fork extension 30B resides above the lower fork extension 30A.

As shown in FIG. 8, the upper handle 20A and lower handle 20B can be moved away from each by the user using a single hand such that the upper and lower fork extensions 30B, 30A move apart from each other to an open position. A first slit gauze G₁ can be placed on the first and second lower prongs 34A, 36A of the lower fork extension 30A with a slit S on a folded side FS₁ of the first slit gauze G₁ residing between the first and second lower prongs 34A, 36A when the upper and lower fork extensions 30B, 30A are in the open position. To facilitate proper placement of the gauze G₁, the folded side FS₁ of the first slit gauze G₁ can be positioned against the blunt ends of the first and second lower prongs 34A, 36A. The user can then move the upper handle 20A and lower handle 20B toward each other such that the upper and lower fork extensions 30B, 30A move toward each other to a closed position so that the upper and lower fork extensions 30B, 30A hold the first slit gauze G₁ therebetween.

If a second gauze is desired, a second slit gauze G₂ can be on the first and second upper prongs 34B, 36B of the upper fork extension 30B with a slit S₂ on a folded side FS₂ of the second slit gauze G₂ residing between the first and second upper prongs 34B, 36B when the upper and lower fork extensions are in the open position as shown in FIG. 10. Similarly, the folded side FS₂ of the second slit gauze G₂ can be positioned against the blunt ends of the first and second upper prongs 34B, 36B. With the second slit gauze G₂ in place, the user can then move the upper handle 20A and lower handle 20B toward each other such that the upper and lower fork extensions 30B, 30A move toward each other to a closed position so that the upper and lower fork extensions 30B, 30A hold the first slit gauze G₁ and the second split gauze G₂ therebetween.

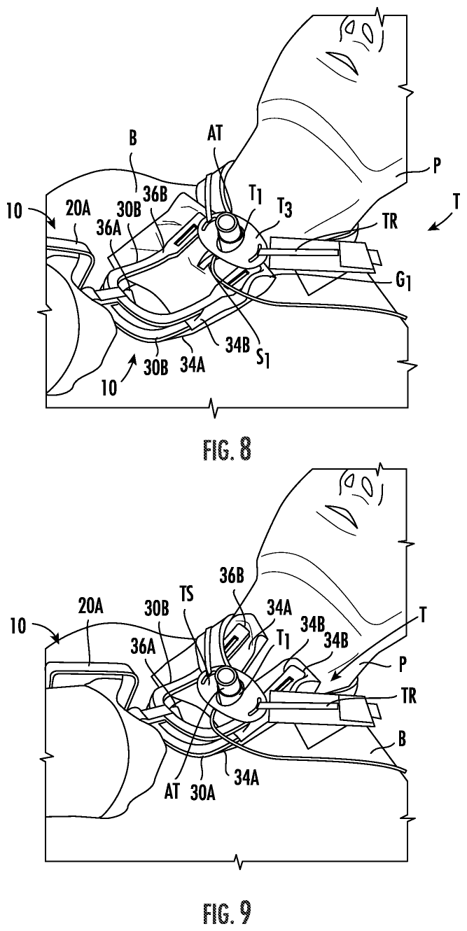

Once the one or more gauzes are hold in a proper position by the gauze placement apparatus 10, the first slit gauze G₁ (and/or the second split gauze G₂ if used) can be positioned on a patient P around tracheostomy equipment T using the gauze placement apparatus 10. For example, the shape of the first and second lower prongs 34A, 36A and first and second upper prongs 34B, 36B can facilitate insertion of the first slit gauze G₁ (and/or the second split gauze G₂ if used) between a portion of the tracheostomy equipment T and the patient P. As shown in FIGS. 8-9, with the user's hand holding the handles 20A, 20B above the fork extensions 30A, 30B, the first and second lower prongs 34A, 36A and first and second upper prongs 34B, 36B with the first slit gauze G₁ thereon can be inserted beneath a shield TS of the tracheostomy equipment T and straps TR helping to hold the tracheostomy equipment T in place. The slit S₁ of the slit gauze G₁ can be aligned with a tube T₁ in which an annular tube AT is inserted into the body B of the patient P.

The user can position the first slit gauze G₁ in place using the gauze placement apparatus 10 as shown in FIG. 9 by the user moving the handles 20A, 20B downward and forward so that the first and second lower prongs 34A, 36A and first and second upper prongs 34B, 36B move forward and slightly upward placing the slit S₁ of the slit gauze G₁ around a portion of the tracheostomy equipment T and the slit gauze G₁ on the patient P between the shield TS of the tracheostomy equipment T and the body B of the patient P. The user can then separate the handles 20A, 20B to move the fork extensions 30B, 30B (and the first and second lower prongs 34A, 36A and first and second upper prongs 34B, 36B) to an open position to release the slit gauze G₁. The user can then the handles 20A, 20B upward and backward so that the first and second lower prongs 34A, 36A and first and second upper prongs 34B, 36B move backward and slightly downward to remove the gauze placement apparatus 10 from around the tracheostomy equipment T leaving the slit gauze G₁ in place.

Referring to FIG. 11, another embodiment of a gauze placement apparatus, generally designated 110, is provided that can be used to insert gauzes on a patient between medical equipment and the body of the patient around a portion of the medical equipment. The gauze placement apparatus 110 comprises a pair of handles 120A, 120B and a pair of fork extensions 130A, 130B connected to the respective handles 120A, 120B. One handle 120A is positioned above the other handle 120B. The pair of fork extensions 130A, 130B can be positioned so each fork extension 130A, 130B is vertically in line with the other. Each fork extension 130A, 130B can also include a fork portion 132A, 132B and a pair of prongs 134A, 136A and 134B, 136B extending from the fork portion 132A, 132B. The pair of prongs 134A, 136A and 134B, 136B on each forked extension 130A, 130B can be horizontally in line with the other. The pair of handles 120A, 120B can be described as a first handle 120A and a second handle 120B. Similarly, the pair of forked extensions 130A, 130B can be considered a first forked extension 130A and a second forked extension 130B. The pair of handles 120A, 120B and the pair of forked extensions 130A, 130B can be pivotally connected such that the first handle 120A can reside above the second handle 120B and the first forked extension 130A can reside above the second forked extension 130B. As the pair of handles 120A, 120B is squeezed together, the pivotal connection allows the pair of forked extensions 130A, 130B to be squeezed together as well. As the pair of handles 120A, 120B is separated, the pair of forked extensions 130A, 130B is separated as a result.

These and other modifications and variations to the present subject matter may be practiced by those of ordinary skill in the art, without departing from the spirit and scope of the present subject matter, which is more particularly set forth herein above and any appending claims. In addition, it should be understood the aspects of the various embodiments may be interchanged either in whole or in part. Furthermore, those of ordinary skill in the art will appreciate that the foregoing description is by way of example only, and is not intended to limit the present subject matter.

1. A gauze placement apparatus comprising:
an upper handle connected to a lower fork extension;
a lower handle connected to an upper fork extension;
the lower fork extension comprising a fork portion and first and second lower prongs aligned with each other in a second direction that is transverse to a first direction in which the upper handle is oriented;
the upper fork extension comprising a fork portion and first and second upper prongs aligned with each other in the second direction that is transverse to the first direction in which the lower handle is oriented;
the upper handle and the lower fork extension being pivotally connected to the lower handle and the upper fork extension such that the upper handle resides above the lower handle and the upper fork extension resides above the lower fork extension so that, when the upper handle and lower handle are moved in a diverging direction, the upper and lower fork extensions move apart from each other to an open position and, when the upper handle and lower handle are moved in a converging direction, the upper and lower fork extensions move toward each other to a closed position; and
the first and second lower prongs of the lower fork extension configured to receive a first folded gauze with a slit on a folded side of the folded gauze such that the slit resides between the first and second lower prongs and the upper and lower fork extensions configured to hold the first folded gauze when in the closed position.

2. The gauze placement apparatus according to claim 1, wherein the first and second upper prongs of the upper fork extension are configured to receive a second folded gauze with a slit on a folded side of the second folded gauze such that the slit resides between the first and second upper prongs and the upper and lower fork extensions configured to hold the second folded gauze when in the closed position.

3. The gauze placement apparatus according to claim 1, wherein the first and second upper prongs of the upper fork extension each comprise a shank and an insertion tip and the first and second lower prongs of the lower fork extension each comprise a shank an insertion tip and wherein the first upper prong aligns with the first lower prong in the first direction transverse to the second direction in which the first and second upper prongs are aligned and the second upper prong aligns with the second lower prong in the first direction transverse to the second direction in which the first and second upper prongs are aligned.

4. The gauze placement apparatus according to claim 3, wherein the insertion tip of the first upper prong and the insertion tip of the first lower prong converge in the closed position and the insertion tip of the second upper prong and the insertion tip of the second lower prong converge in the closed position to facilitate insertion of one or more gauzes between a patient and a portion of medical equipment.

5. The gauze placement apparatus according to claim 4, wherein the shank of the first upper prong and the shank of the first lower prong are spaced apart in the closed position and the shank of the second upper prong and the shank of the second lower prong are spaced apart in the closed position such that a separation space is formed between portions of the upper fork extension and the lower fork extension when the upper and lower extensions are in the closed position.

6. The gauze placement apparatus according to claim 3, wherein the first and second lower prongs of the lower fork extension are separated by a distance large enough to permit the first and second lower prongs to be inserted on either side of a tube inserted into a patient and the first and second upper prongs of the upper fork extension are separated by a distance large enough to permit the first and second lower prongs to be inserted on either side of a tube inserted into a body of a patient for placement of one or more gauzes around the tube.

7. The gauze placement apparatus according to claim 1, wherein the first and second lower prongs of the lower fork extension and the first and second upper prongs of the upper fork extension curve upward to facilitate insertion of one or more gauzes around a tube inserted into a body of a patient.

8. The gauze placement apparatus according to claim 7, wherein the first and second lower prongs of the lower fork extension each has an inner radius of curvature and an outer radius of curvature and the first and second upper prongs of the upper fork extension each has an inner radius of curvature and an outer radius of curvature.

9. The gauze placement apparatus according to claim 8, wherein the inner radii of curvature of the first and second lower prongs are greater than the outer radii of curvature of first and second lower prongs such that upper and lower outer surfaces of first and second lower prongs converge toward blunt ends of the respective first and second lower prongs; and
wherein the inner radii of curvature of the first and second upper prongs are greater than the outer radii of curvature of first and second upper prongs such that upper and lower outer surfaces of first and second upper prongs converge toward blunt ends of the respective first and second upper prongs.

10. The gauze placement apparatus according to claim 8, wherein the outer radii of curvature of first and second upper prongs are greater than the outer radii of curvature of first and second lower prongs such that the first and second lower prongs converge toward the respective first and second upper prongs when the upper and lower fork extensions are in the closed position.

11. The gauze placement apparatus according to claim 8, wherein the outer radii of curvature of first and second lower prongs of the lower fork extensions and lengths of the first and second upper prongs of the upper fork extensions and the first and second lower prongs of the lower fork extensions are such that the blunt ends of first and second lower prongs and the first and second upper prongs are insertable under portions of a tracheostomy equipment and a slit gauze on the upper prongs is placeable around a tube of the tracheostomy equipment with the base of the slit against the tube without the fork portion of the lower fork extension or the lower handle abutting the body of the patient.

12. The gauze placement apparatus according to claim 11, wherein the first and second lower prongs curve upward from the fork portion of the lower fork extension such that a vertical distance from a plane that extends from an outer surface of a base of the fork portion of the lower fork extension to the blunt ends of first and second lower prongs is about 2 inches or more.

13. The gauze placement apparatus according to claim 11, wherein the first and second lower prongs curve upward from the fork portion of the lower fork extension such that a vertical distance from a plane that extends from an outer surface of a base of the fork portion of the lower fork extension to the blunt ends of first and second lower prongs is between about 2 inches and about 4 inches.

14. The gauze placement apparatus according to claim 1, wherein the handles comprise hand inserts to move the upper and lower fork extensions between the open position to the closed position.

15. A gauze placement apparatus comprising:
an upper handle connected to a lower fork extension;
a lower handle connected to an upper fork extension;
the lower fork extension comprising a fork portion and first and second lower prongs aligned with each other in a second direction that is transverse to a first direction in which the upper handle is oriented;
the upper fork extension comprising a fork portion and first and second upper prongs aligned with each other in the second direction that is transverse to the first direction in which the lower handle is oriented;
the upper handle and the lower fork extension being pivotally connected to the lower handle and the upper fork extension such that the upper handle resides above the lower handle and the upper fork extension resides above the lower fork extension so that, when the upper handle and lower handle are moved in a diverging direction, the upper and lower fork extensions move apart from each other to an open position and, when the upper handle and lower handle are moved in a converging direction, the upper and lower fork extensions move toward each other to a closed position;
the first and second lower prongs of the lower fork extension configured to receive a first folded gauze with a slit on a folded side of the folded gauze such that the slit resides between the first and second lower prongs and the upper and lower fork extensions configured to hold the first folded gauze when in the closed position; and
the first and second lower prongs of the lower fork extension and the first and second upper prongs of the upper fork extension curve upward to facilitate insertion of one or more gauzes between a portion of medical equipment and a body of a patient.

16. The gauze placement apparatus according to claim 15, wherein the first and second lower prongs of the lower fork extension each has an inner radius of curvature and an outer radius of curvature and the first and second upper prongs of the upper fork extension each has an inner radius of curvature and an outer radius of curvature.

17. The gauze placement apparatus according to claim 16, wherein the inner radii of curvature of the first and second lower prongs are greater than the outer radii of curvature of first and second lower prongs such that upper and lower outer surfaces of first and second lower prongs converge toward blunt ends of the respective first and second lower prongs; and
wherein the inner radii of curvature of the first and second upper prongs are greater than the outer radii of curvature of first and second upper prongs such that upper and lower outer surfaces of first and second upper prongs converge toward blunt ends of the respective first and second upper prongs.

18. The gauze placement apparatus according to claim 17, wherein the outer radii of curvature of first and second upper prongs are greater than the outer radii of curvature of first and second lower prongs such that the first and second lower prongs converge toward the respective first and second upper prongs when the upper and lower fork extensions are in the closed position.

19. A method of placing a gauze on a patient, the method comprising:
providing a gauze placement apparatus comprising:
an upper handle connected to a lower fork extension, the lower fork extension comprises a fork portion and first and second lower prongs aligned with each other in a second direction that is transverse to a first direction in which the upper handle is oriented;
a lower handle connected to an upper fork extension, the upper fork extension comprises a fork portion and first and second upper prongs aligned with each other in a second direction that is transverse to a first direction in which the upper handle is oriented;
the upper handle and the lower fork extension being pivotally connected to the lower handle and the upper fork extension such that the upper handle resides above the lower handle and the upper fork extension resides above the lower fork extension;
moving the upper handle and lower handle away from each such that the upper and lower fork extensions move apart from each other to an open position;
placing a first slit gauze on the first and second lower prongs of the lower fork extension with a slit on a folded side of the first slit gauze residing between the first and second lower prongs when the upper and lower fork extensions are in the open position;
moving the upper handle and lower handle toward each other such that the upper and lower fork extensions move toward each other to a closed position so that the upper and lower fork extensions hold the first slit gauze therebetween; and
positioning the first slit gauze on first and second lower prongs on a patient between the patient and medical equipment using the gauze placement apparatus.

20. The method according to claim 19, further comprising:
placing a second slit gauze on the first and second upper prongs of the upper fork extension with a slit on a folded side of the second slit gauze residing between the first and second upper prongs when the upper and lower fork extensions are in the open position;
moving the upper handle and lower handle toward each other such that the upper and lower fork extensions move toward each other to the closed position so that the upper and lower fork extensions hold the second slit gauze and the first slit gauze therebetween;
positioning the first slit gauze on first and second lower prongs and the second slit gauze on first and second upper prongs on a patient between the patient and medical equipment using the gauze placement apparatus.
